(19) United States
(12) Patent Application Publication
(10) Pub. No.: US 2024/0299159 A1
(43) Pub. Date: Sep. 12, 2024

(54) ARTIFICAL EYE LENS CAPABLE OF DESORPTING OR ADDING AN OPTICAL LENS AND INSERTION METHOD THEREOF

(71) Applicant: WELLC LLC, Busan (KR)

(72) Inventors: Ki Sung PARK, Busan (KR); Jin A. PARK, Hwaseong-si (KR)

(73) Assignee: WELLC LLC, Busan (KR)

(21) Appl. No.: 18/488,775

(22) Filed: Oct. 17, 2023

(30) Foreign Application Priority Data
Mar. 8, 2023 (KR) 10-2023-0030798

(51) Int. Cl. A61F 2/16

(52) U.S. Cl. CPC A61F 2/1648; A61F 2002/169; A61F 2002/16902; A61F 2220/0025

(57) ABSTRACT

The present disclosure relates to an intraocular lens. An intraocular lens in accordance with some embodiments of the present disclosure comprises a first optical portion including an optical lens and a support portion configured to be coupled with the first optical portion, wherein the first optical portion may be formed to be detachable from the support portion in a state where the support portion is inserted into an eye of a user.

### TECHNICAL FIELD

The present disclosure relates to an intraocular lens from or to which an optical lens can be detached or added and a method of inserting the same.

More particularly, the present disclosure relates to an intraocular lens that can improve optical errors in the intraocular lens more safely through detachment or additional mounting of an optical lens without extracting the intraocular lens itself inserted into the eye after cataract surgery, and a method of inserting the same.

In addition, the present disclosure relates to an intraocular lens having a predetermined number of through-holes (openings) formed therein through which surgical instruments for simple retinal surgery can be inserted.

### BACKGROUND

This patent application was supported by a grant from the Korea Institute for Advancement of Technology (KIAT) of the Ministry of Trade, Industry and Energy (MOTIE). The project number is B0080406003265, the research project is Support for Globalization of Ophthalmic Medical Devices, and the research project name is an individual national application for ARTIFICIAL EYE LENS CAPABLE OF DESORPTING OR ADDING AN OPTICAL LENS AND INSERTION METHOD THEREOF. The contribution ratio is 1/1, and the name of the institution that carries out the project is Chosun University Industry-University Cooperation Foundation. The research period is 2023. 4. 1˜2023. 12. 31.

A conventional intraocular lens is a unitary product configured in the form of a central part including an optical lens and a haptic part that supports the central part.

In this case, if there arises a need to replace the intraocular lens, a new intraocular lens must be inserted to resolve this. The cases where there is a need to replace the intraocular lens include a case where a refractive error occurs after cataract surgery, a case where it is necessary to supplement the function of the intraocular lens from monofocal to multifocal or to modify from multifocal to monofocal, a case where the intraocular lens dislocates from the lens capsule, a case where opacification occurs in the intraocular lens, a case where bullous keratopathy (corneal endothelial reduction or cystic macular edema) occurs due to the intraocular lens, a case where the intraocular lens is lost due to trauma, and the like.

In general, the process of extracting the existing intraocular lens is preceded in order to insert a new intraocular lens, and this method is very difficult to use if the intraocular lens and lens capsule are stuck together, and is also impossible if secondary cataract surgery has been performed.

Therefore, there has been a sufficient unmet need for an intraocular lens whose optical functions can be easily improved and supplemented through the replacement or addition of optical lenses.

### SUMMARY

Aspects of the disclosure an intraocular lens from or to which an optical lens can be detached or added and a method of inserting the same.

Specifically, an object to be achieved by the present disclosure is to provide an intraocular lens that can improve the function of an optical lens of the intraocular lens more safely through the detachment or additional mounting of the optical lens without extracting the intraocular lens itself that has already been inserted into the eye, and a method of inserting the same.

Another aspects of the disclosure an intraocular lens having a predetermined number of through-holes (openings) formed therein through which surgical instruments for simple retinal surgery can be inserted.

The objects of the present disclosure are not limited to the objects mentioned above, and other objects and advantages of the present disclosure that have not been mentioned can be understood by the following description and will be more clearly understood by the embodiments of the present disclosure. Further, it will be readily appreciated that the objects and advantages of the present disclosure may be realized by the means set forth in the claims and combinations thereof.

An intraocular lens in accordance with some embodiments of the present disclosure comprises a first optical portion including an optical lens and a support portion configured to be coupled with the first optical portion, wherein the first optical portion may be formed to be detachable from the support portion in a state where the support portion is inserted into an eye of a user.

In addition, the first optical portion further comprises an access body, the support portion comprises a connecting body, the access body and the connecting body may be coupled with each other, and the access body and the connecting body may be the same in number, which is at least two or more.

Further, the support portion and the first optical portion may have shapes corresponding to each other so as to be coupled with each other.

Moreover, the support portion may comprise a predetermined number of through-holes.

Furthermore, the intraocular lens may further comprise sealing buttons configured to be inserted into the through-holes formed in the support portion and perform a sealing function.

An intraocular lens in accordance with some other embodiments of the present disclosure comprises a first optical portion including an optical lens, a support portion configured to be coupled with the first optical portion, and an additional optical portion configured to be coupled to the support portion, wherein the first optical portion and the additional optical portion may be formed to be detachable from the support portion in a state where the support portion is inserted into an eye of a user, and the first optical portion and the additional optical portion may be coupled, respectively, to sides that are symmetrical to each other in the support portion.

An intraocular lens in accordance with some other embodiments of the present disclosure comprises a second optical portion including an optical lens, and a whole portion configured to be coupled with the second optical portion and including a basic lens formed on a side in contact with the second optical portion, wherein the second optical portion may be formed to be detachable from the whole portion in a state where the whole portion is inserted into an eye of a user.

Further, the second optical portion further comprises an access body, the whole portion comprises a connecting body, the access body and the connecting body may be coupled with each other, and the access body and the connecting body may be the same in number, which is at least two or more.

In addition, the whole portion may comprise a predetermined number of through-holes.

Moreover, the intraocular lens may further comprise sealing buttons insertable into the through-holes formed in the whole portion.

The present disclosure has the novel effect of enabling the detachment or addition of an optical lens after inserting an intraocular lens into the eye.

Specifically, the present disclosure has the novel effect of making it possible to perform the replacement of an intraocular lens more safely through the detachment or additional mounting of an optical lens without extracting the intraocular lens itself after insertion of the intraocular lens.

In addition, the present disclosure makes it possible to insert surgical instruments via through-holes by having a predetermined number of through-holes (openings) formed in the intraocular lens. Through this, there is a remarkable effect that simple retinal surgery can be performed in a short period of time without bleeding.

In addition to the contents described above, specific effects of the present disclosure will be described together while describing the following specific details for carrying out the present disclosure.

### BRIEF DESCRIPTION OF THE DRAWINGS

FIGS. 1a and 1b are diagrams for describing problems with conventional intraocular lenses;

FIGS. 2a to 2d illustrate prefabricated intraocular lenses in accordance with some embodiments of the present disclosure;

FIG. 3 illustrates prefabricated intraocular lenses according to the type of optical lens of an optical portion;

FIG. 4 illustrates a double-sided prefabricated intraocular lens in accordance with some embodiments of the present disclosure;

FIGS. 5a to 5e illustrate functional intraocular lenses in accordance with some embodiments of the present disclosure;

FIGS. 6a and 6b are diagrams for describing a connection method between an access body and a connecting body in accordance with some embodiments of the present disclosure;

FIG. 7 is a diagram for describing a sealing button and a through-hole in accordance with some embodiments of the present disclosure;

FIGS. 8a to 8c are flowcharts illustrating a method of inserting intraocular lenses into eyes in accordance with some embodiments of the present disclosure; and

FIG. 8d is a flowchart illustrating a method of retinal surgery via through-holes and a method of sealing the through-holes in accordance with some embodiments of the present disclosure.

### DETAILED DESCRIPTION OF EMBODIMENTS

The terms or words used in the present description and the claims should not be construed as limited to their ordinary or lexical meanings. They should be construed as the meaning and concept in line with the technical idea of the present disclosure based on the principle that the inventor can define the concept of terms or words in order to describe his/her own invention in the best possible way. Further, since the embodiments described herein and the configurations illustrated in the drawings are merely example embodiments in which the present disclosure is realized and do not represent all the technical ideas of the present disclosure, it should be understood that there may be various equivalents, variations, and applicable examples that can replace them at the time of filing this application.

Although terms such as first, second, A, B, etc. used in the present description and the claims may be used to describe various components, the components should not be limited by these terms. These terms are used only for the purpose of distinguishing one component from another. For example, a first component may be referred to as a second component, and similarly, a second component may be referred to as a first component, without departing from the scope of the present disclosure. The term ‘and/or’ includes a combination of a plurality of related listed items or any item of the plurality of related listed items.

The terms used in the present description and the claims are merely used to describe particular embodiments and are not intended to limit the present disclosure. Singular expressions include plural expressions unless the context explicitly indicates otherwise. In the present application, terms such as “include,” “have,” etc. should be understood as not precluding the possibility of existence or addition of features, numbers, steps, operations, components, parts, or combinations thereof described herein.

Unless defined otherwise, all terms used herein, including technical or scientific terms, have the same meaning as commonly understood by those of ordinary skill in the art to which the present disclosure pertains.

Terms such as those defined in commonly used dictionaries should be construed as having a meaning consistent with the meaning in the context of the relevant art, and are not to be construed in an ideal or excessively formal sense unless explicitly defined in the present application. In addition, each configuration, procedure, process, method, or the like included in each embodiment of the present disclosure may be shared to the extent that they are not technically contradictory to each other.

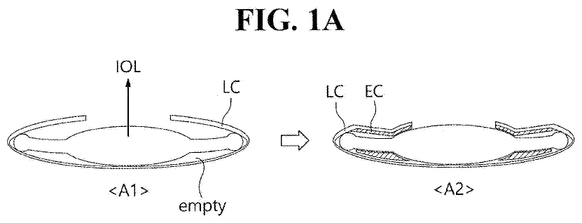

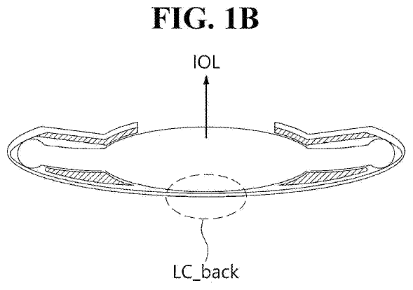

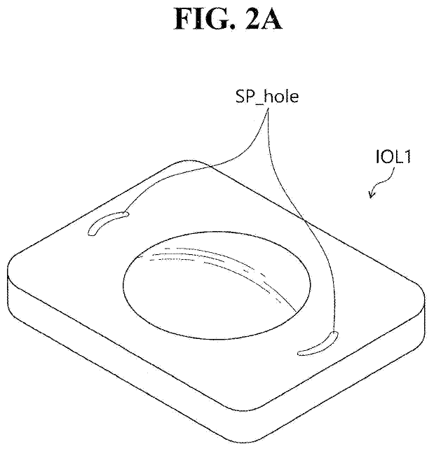

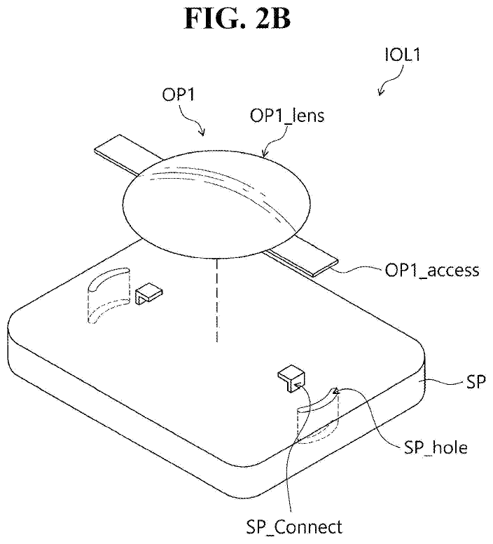

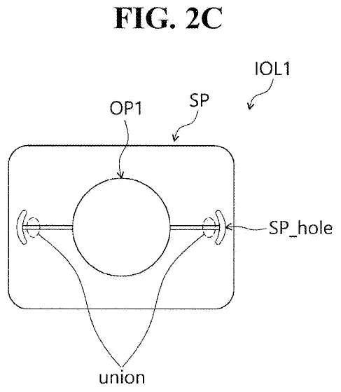

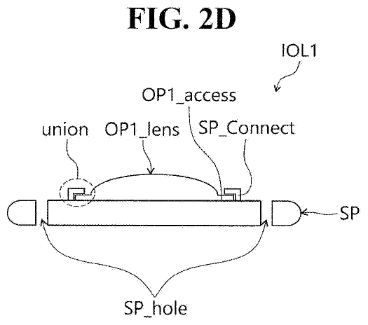

Hereinafter, problems with conventional intraocular lenses will be described with reference to FIGS. 1a and 1b, and an intraocular lens and a method of inserting the same in accordance with some embodiments of the present disclosure will be described with reference to FIGS. 2a to 8d.

FIGS. 1a and 1b are diagrams for describing problems with conventional intraocular lenses.

Referring to FIGS. 1a and 1b, a conventional intraocular lens (hereinafter referred to as “IOL”) is a unitary product consisting of a central part including an optical lens and a haptic part that supports the central part.

In this case, if there arises a need to replace the intraocular lens IOL, anew intraocular lens IOL must be inserted to resolve this. The cases where there is a need to replace the intraocular lens IOL include a case where a refractive error occurs after cataract surgery, a case where it is necessary to supplement the function of the intraocular lens IOL from monofocal to multifocal or to modify from multifocal to monofocal, a case where the intraocular lens IOL dislocates from the lens capsule (hereinafter referred to as “LC”), a case where opacification occurs in the intraocular lens IOL, a case where bullous keratopathy (corneal endothelial reduction or cystic macular edema) occurs due to the intraocular lens IOL, a case where the intraocular lens IOL is lost due to trauma, and the like.

In general, the process of extracting the existing intraocular lens IOL is preceded in order to insert a new intraocular lens IOL, and this method is very difficult to use if the intraocular lens IOL and lens capsule LC are stuck together, and is also impossible if secondary cataract surgery has been performed.

Described specifically, first, if only a few months (e.g., three months) have passed since the insertion of an intraocular lens IOL and the lens capsule LC has not been stuck with the intraocular lens IOL (see <A1> in FIG. 1a), it is necessary to cut the intraocular lens IOL contained within the lens capsule LC into several pieces and then extract them from the eye, insert a new intraocular lens IOL, and then stitch the intraocular lens IOL extraction site, in order to replace the intraocular lens IOL. This is a complex and time-consuming surgical procedure, and there is also the possibility of touching the endothelial cells of the cornea or rupturing the lens capsule LC.

Next, approximately three to six months after the intraocular lens IOL is inserted into the lens capsule LC, epithelial cells EC proliferate in the empty space of the lens capsule LC and firmly stick to the intraocular lens IOL (see <A2> in FIG. 1a).

At this time, because the inserted intraocular lens IOL is firmly fixed to the lens capsule LC, it is very difficult to extract and replace the intraocular lens IOL that is stuck with the lens capsule LC. When extracting such an intraocular lens IOL, the surgical procedure is complicated and the surgery takes a long time, as well as the lens capsule LC is easily torn when attempting to separate the lens capsule LC stuck with the intraocular lens IOL, and if the lens capsule LC is torn, not only does the vitreous body that fills the retinal area behind the lens capsule LC leak out, but it is also impossible to insert a new intraocular lens IOL into the lens capsule LC after extracting the intraocular lens IOL.

Accordingly, if it turned out to be impossible to insert an intraocular lens IOL into the lens capsule for the reasons described above, this problem was previously resolved using an anterior intraocular lens IOL insertion technique in which a new intraocular lens IOL was inserted in front of the iris, and a posterior intraocular lens IOL insertion technique in which a new intraocular lens IOL was inserted behind the iris.

However, the anterior intraocular lens IOL insertion technique is highly likely to cause side effects such as damage to the corneal endothelium and blockage of the anterior angle that can lead to glaucoma. In addition, the posterior intraocular lens IOL insertion technique is a method of connecting both ends of the haptic part that supports the optical portion of the intraocular lens IOL with sutures and then sewing both ends of the haptic part to the adjacent sclera, and has disadvantages that the surgical procedure is complicated, the surgery takes a long time, and visual discomfort occurs because it cannot be fixed like an intraocular lens IOL in the lens capsule LC after surgery.

To resolve the above disadvantages, there is a surgical procedure in which a support portion for an intraocular lens IOL is created and the haptic part is melted with a thermal cautery and is fixed to a plate-like flenge, which, however, is not a commonly practiced surgical method.

If secondary cataract surgery that removes the lens capsule LC stuck to the back of the intraocular lens IOL, i.e., the posterior lens capsule LC_back with a laser is performed after cataract surgery, the replacement of the intraocular lens IOL will be completely impossible.

Referring to FIG. 1b, secondary cataract refers to a symptom in which epithelial cells proliferate and opacification occurs in the lens capsule LC behind the lens, i.e., in the posterior lens capsule LC_back. To resolve this, the posterior lens capsule LC_back is burned and eliminated with a laser during secondary cataract surgery. At this time, if the intraocular lens IOL is removed because there is a need for replacement of the intraocular lens IOL, leakage of the highly viscous vitreous body that fills the retinal area behind the lens capsule LC occurs inevitably.

Therefore, if secondary cataract surgery has been performed after inserting a conventional intraocular lens IOL, replacement of the intraocular lens IOL is not possible.

In addition, if there occurs a small amount of vitreous floaters, minute vitreous opacification, or minute retinal tears within the existing retina, there has been no method to simply resolve this until now. Since all of these symptoms had to be treated only through the procedure of conventional retinal surgery, ophthalmologists who were not retina specialists were unable to treat them.

Therefore, there has been a sufficient need for intraocular lens technology to solve these problems and inconveniences.

In the following, an intraocular lens and a method of inserting the same that can easily solve the above problems and inconveniences will be described.

FIGS. 2a to 2d illustrate prefabricated intraocular lenses in accordance with some embodiments of the present disclosure.

The intraocular lens IOL1 shown in FIGS. 2a to 2d is an intraocular lens whose optical portion can be detached from a support portion, and may be referred to as a prefabricated intraocular lens. For convenience of description below, the intraocular lens IOL1 shown in FIGS. 2a to 2d will be referred to by the same name as the prefabricated intraocular lens IOL1. The material of the prefabricated intraocular lens IOL1 may include biocompatible materials, for example, silicone and/or polymethyl methacrylate (PMMA), but embodiments of the present disclosure are not limited thereto.

FIG. 2a is a perspective view of the prefabricated intraocular lens IOL1, FIG. 2b is an exploded perspective view of the prefabricated intraocular lens IOL1, FIG. 2c is a plan view (top view) of the prefabricated intraocular lens IOL1, and FIG. 2d is a front view of the prefabricated intraocular lens IOL1.

Referring to FIGS. 2a to 2d, the prefabricated intraocular lens IOL1 may include an optical portion (hereinafter referred to as “OP”) and a support portion (hereinafter referred to as “SP”).

The optical portion OP of the prefabricated intraocular lens IOL1 may be referred to as a first optical portion OP1 so as to be distinguished from the optical portion OP of a functional intraocular lens IOL_2, which will be described later. For convenience of description below, the optical portion included in the prefabricated intraocular lens IOL1 will be described by being referred to as the first optical portion OP1, and the optical portion included in the functional intraocular lens IOL_2 will be described by being referred to as a second optical portion OP2.

The first optical portion OP1 may determine the optical properties of the prefabricated intraocular lens IOL1. In other words, the first optical portion OP1 may serve to change the traveling direction of light by collecting or focusing the light onto the prefabricated intraocular lens IOL1.

As some examples, the first optical portion OP1 may include an optical lens OPT_lens. In this case, the optical lens OP1_lens may have various parameter values. The parameters of the optical lens OP1_lens may include the type, focal length, refractive index, progression angle, diameter, thickness, and the like of the optical lens OP1_lens, but embodiments of the present disclosure are not limited thereto. The types of the optical lens OP1_lens may include a biconvex lens, a single-sided convex lens, a bending concave lens that is curved like the shape of the cornea, and the like, but embodiments of the present disclosure are not limited thereto.

As some examples, the first optical portion OP1 may include an access body OPT_access for being connected with the support portion SP.

Although FIG. 2b shows that the access body OPT_access has the shape of a bar and the number of access bodies OPT_access is two, this is merely for the convenience of description. That is, the shape and number of the access bodies OPT_access are not limited to those shown in FIG. 2b and can be freely modified.

In this case, the access body OPT_access may be connected with a connecting body SP_connect of the support portion SP. In other words, the access body OPT_access of the first optical portion OP1 may be connected with the connecting body SP_connect of the support portion SP, thereby forming a union. The specific method in which the access body OPT_access and the connecting body SP_connect are connected will be described later.

The support portion SP is connected with the first optical portion OP1 and serves to support and sustain the first optical portion OPT.

Although FIGS. 2a to 2d shows that the support portion SP has a rectangular parallelepiped shape with rounded corners, embodiments of the present disclosure are not limited thereto, and as a matter of course, the shape of the support portion SP may have a variety of shapes, such as a cube with rounded corners, a cylinder, or a cone.

As some examples, the support portion SP may include the connecting body SP_connect for being connected with the first optical portion OP1.

Although FIG. 2b shows that the connecting body SP_connect has a, shape and the number of connecting bodies SP_connect is two, this is merely for the convenience of description. That is, the shape and number of the connecting bodies SP_connect are not limited to those shown in FIG. 2b and can be freely modified.

As some examples, the support portion SP may include a predetermined number of through-holes SP_hole. The through-holes SP_hole may be utilized as a space into which surgical instruments for simple retinal surgery can be inserted. Simple retinal surgery may include treatment of vitreous floaters, small-scale vitrectomy, treatment of minute retinal tears, and the like, but embodiments of the present disclosure are not limited thereto. In this case, the diameter of the through-holes SP_hole may be 2 mm or less, and the diameter of the surgical instruments to be inserted into the through-holes SP_hole may be 1 mm or less, but embodiments of the present disclosure are not limited thereto.

Although FIGS. 2a to 2c show that the through-holes SP_hole have an arc shape, and the number of through-holes SP_hole is two, this is merely for the convenience of description. That is, the shape and number of through-holes SP_hole are not limited to those shown in FIGS. 2b and 2c and can be freely modified.

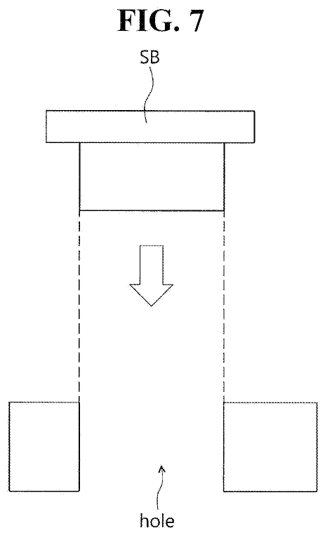

Although not shown in FIGS. 2a to 2d, the prefabricated intraocular lens IOL1 of the present disclosure may further include sealing buttons to be inserted into the through-holes SP_hole. The sealing buttons will be described in more detail later in FIG. 7.

Such a prefabricated intraocular lens IOL1 has a novel effect of enabling the detachment and replacement of the optical portion (first optical portion, OP1) after being inserted into the eye. That is, the prefabricated intraocular lens IOL1 of the present disclosure makes it possible to perform the replacement of the intraocular lens more safely through the process of detaching and replacing the first optical portion OP1 without extracting the intraocular lens itself after being inserted into the eye.

Meanwhile, the first optical portion OP1 and the support portion SP may have shapes corresponding to each other so that they can be coupled to each other. For example, the support portion SP may be formed with a curvature corresponding to the curvature of the optical lens OP1_lens of the first optical portion OP1.

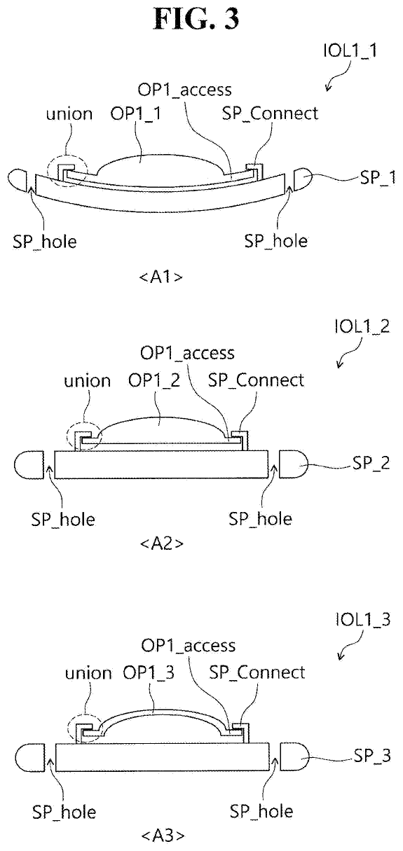

In the following, prefabricated intraocular lenses IOL1 including a support portion SP having a different shape according to the type of the optical lens OP1_lens of the first optical portion OP1 will be described with reference to FIG. 3.

FIG. 3 illustrates prefabricated intraocular lenses according to the type of the optical lens of the optical portion. That is, FIG. 3 shows front views of prefabricated intraocular lenses IOL1_1, IOL1_2, and IOL1_3 including different optical lenses.

Specifically, <A1> in FIG. 3 shows a prefabricated intraocular lens IOL1_1 including an optical portion OP1_1 whose optical lens is a biconvex lens, <A2> in FIG. 3 shows a prefabricated intraocular lens IOL1_2 including an optical portion OP1_2 whose optical lens is a one-sided convex lens, and <A3> in FIG. 3 shows a prefabricated intraocular lens IOL1_3 including an optical portion OP1_3 whose optical lens is a bending concave lens.

Referring to <A1> in FIG. 3, the prefabricated intraocular lens IOL1_1 shown in <A1> of FIG. 3 includes an optical portion OP1_1 whose optical lens is a biconvex lens. Therefore, the support portion SP_1 of the corresponding prefabricated intraocular lens IOL1_1 may have a shape corresponding to the optical portion OP1_1 in order to be easily coupled with the optical portion OP1_1, as shown in <A1> of FIG. 3. That is, the support portion SP_1 may have a curvature of a predetermined size according to the shape of the optical portion OP1_1.

Referring to <A2> in FIG. 3, the prefabricated intraocular lens IOL1_2 shown in <A2> of FIG. 3 includes an optical portion OP1_2 whose optical lens is a one-sided convex lens. Therefore, the support portion SP_2 of the corresponding prefabricated intraocular lens IOL1_2 may have a shape corresponding to the optical portion OP1_2 in order to be easily coupled with the optical portion OP1_2, as shown in <A2> of FIG. 3. That is, the support portion SP_2 may have a flat shape according to the shape of the optical portion OP1_1.

Referring to <A3> in FIG. 3, the prefabricated intraocular lens IOL1_2 shown in <A3> of FIG. 3 includes an optical portion OP1_3 whose optical lens is a bending concave lens. In this case, the support portion SP_2 may have a flat shape according to the shape of the optical portion OP1_1.

In summary, the support portions SP_1, SP_2, and SP_3 of the prefabricated intraocular lenses IOL1_1, IOL1_2, and IOL1_3 may be formed in shapes corresponding to the first optical portions OP1_1, OP1_2, and OP1_3 so as to be coupled with the first optical portion OP1_1, OP1_2, and OP1_3. For example, the support portion SP_1 may have a predetermined curvature if the optical lens is a biconvex lens as shown in <A1> of FIG. 3, and the support portion SP_1 may have a flat shape if the optical lens is a one-sided convex lens or a bending concave lens as shown in <A2> and <A3> of FIG. 3.

Referring again to FIGS. 2a to 2d, the prefabricated intraocular lens IOL1 may include the form of a double-sided prefabricated intraocular lens.

As some examples, the double-sided prefabricated intraocular lens may include two optical portions to be connected to any two of a plurality of sides of the support portion SP.

For example, the double-sided prefabricated intraocular lens may include a first optical portion OP1 to be connected to atop side of the support portion SP, and an additional optical portion to be connected to the opposite side of the first optical portion OP1, i.e., a bottom side of the support portion SP.

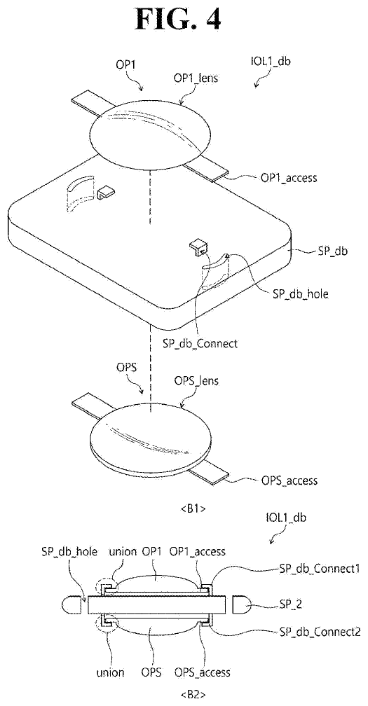

The double-sided prefabricated intraocular lens will be described in more detail with reference to FIG. 4.

FIG. 4 illustrates a double-sided prefabricated intraocular lens in accordance with some embodiments of the present disclosure.

Here, <B1> in FIG. 4 is an exploded perspective view of a double-sided prefabricated intraocular lens IOL1_db, and <B2> in FIG. 4 is a front view of the double-sided prefabricated intraocular lens IOL1_db.

Referring to <B1> in FIG. 4, the double-sided prefabricated intraocular lens IOL1_db may include a first optical portion OP1, an additional optical portion OPS, and a double-sided support portion SP_db.

The first optical portion OP1 has been described above with reference to FIGS. 2a to 2d, and will thus be omitted here.

The additional optical portion OPS may have the same shape as the first optical portion OP1. For example, the additional optical portion OPS may include an access body OPS_access for being connected with the double-sided support portion SP_db.

The additional optical portion OPS may include an optical lens OPS_lens. The parameter values of the optical lens OPS_lens included in the additional optical portion OPS may be the same as or different from the parameter values of the optical lens OP1_lens of the first optical portion OP1. In this case, the parameters of the optical lens OPS_lens may include the type, focal length, refractive index, progression angle, diameter, thickness, and the like of the optical lens OPS_lens, but embodiments of the present disclosure are not limited thereto. The types of the optical lens OPS_lens may include a biconvex lens, a single-sided convex lens, a bending concave lens, and the like, but embodiments of the present disclosure are not limited thereto.

The double-sided support portion SP_db may include through-holes SP_db_hole, a first connecting body SP_db_connect1, and a second connecting body SP_db_connect2. In this case, the first connecting body SP_db_connect1 may be coupled with the first optical portion OP1, thereby forming a union, and the second connecting body SP_db_connect2 may be coupled with the additional optical portion OPS, thereby forming a union.

The through-holes SP_db_hole have been described in detail with reference to FIGS. 2a to 2d, and will thus be omitted here.

The first connecting body SP_db_connect1 and the second connecting body SP_db_connect2 may be formed on different sides of the double-sided support portion SP_db. For example, the first connecting body SP_db_connect1 may be formed on the top side of the double-sided support portion SP_db and connected to the access body OP1_access of the first optical portion OP1, and the second connecting body SP_db_connect2 may be formed on the bottom side of the double-sided support portion SP_db and connected to the access body OPS_access of the additional optical portion OPS.

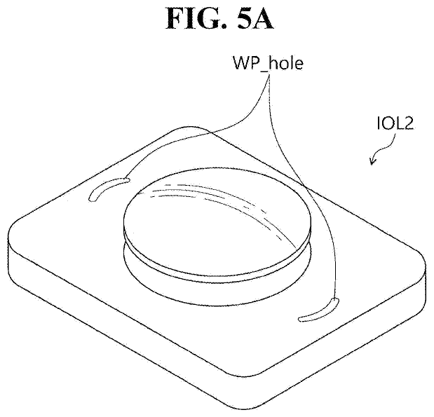

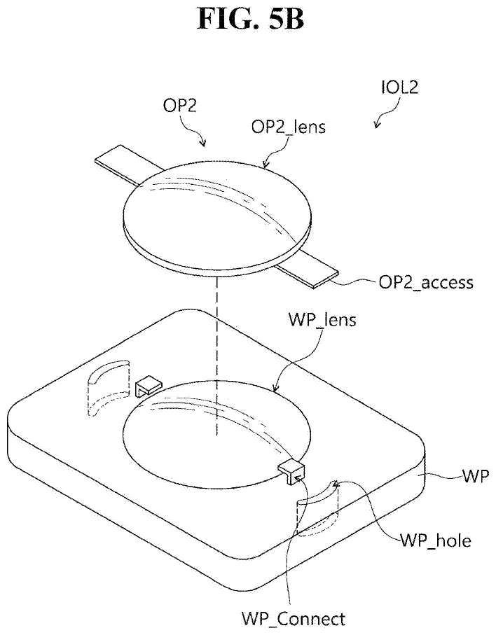

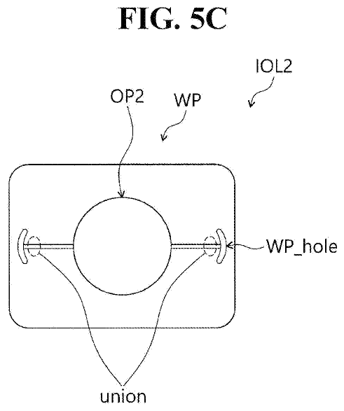

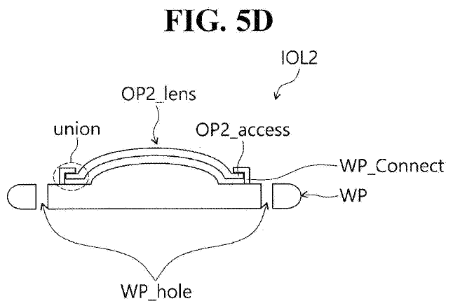

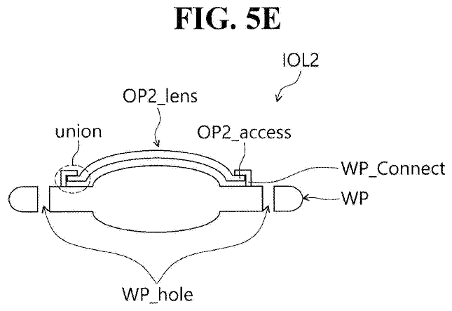

FIGS. 5a to 5e illustrate functional intraocular lenses in accordance with some embodiments of the present disclosure.

The intraocular lens IOL2 shown in FIGS. 5a to 5e is an intraocular lens in which an optical portion can be added to a whole portion that includes a basic lens, and may be referred to as a functional intraocular lens. For convenience of description below, the intraocular lens IOL2 shown in FIGS. 5a to 5e will be referred to by the same name as the functional intraocular lens IOL2. The material of the functional intraocular lens IOL2 may include biocompatible materials, for example, silicone and/or polymethyl methacrylate (PMMA), but embodiments of the present disclosure are not limited thereto.

FIG. 5a is a perspective view of the functional intraocular lens IOL2, FIG. 5b is an exploded perspective view of the functional intraocular lens IOL2, FIG. 5c is a plan view (top view) of the functional intraocular lens IOL2, and FIGS. 5d and 5e are front views of the functional intraocular lens IOL2 in accordance with some embodiments of the present disclosure.

Referring to FIGS. 5a to 5e, the functional intraocular lens IOL2 may include an optical portion OP2 and a whole portion (hereinafter referred to as “WP”).

In this case, the optical portion OP2 of the functional intraocular lens IOL2 may be referred to as a second optical portion OP2 so as to be distinguished from the first optical portion OP1 of the prefabricated intraocular lens IOL_1 described above. In the following, the optical portion OP2 included in the functional intraocular lens IOL2 will be described by being referred to as the second optical portion OP2 for convenience of description.

The functional intraocular lens IOL2 of the present disclosure can perform refractive power correction or improvement, and the like by further using the second optical portion OP2 in addition to the basic lens WP_lens included in the whole portion WP.

That is, the second optical portion OP2 can correct the optical properties of the functional intraocular lens IOL2. In other words, the second optical portion OP2 may assist or rectify the role of changing the traveling direction of light by collecting or focusing the light onto the functional intraocular lens IOL2.

As some examples, the second optical portion OP2 may include an optical lens OP2_lens. In this case, the optical lens OP2_lens may have various parameter values. The parameters of the optical lens OP2_lens may include the type, focal length, refractive index, progression angle, diameter, thickness, and the like of the optical lens OP2_lens, but embodiments of the present disclosure are not limited thereto. The types of the optical lens OP2_lens may include a biconvex lens, a single-sided convex lens, a bending concave lens, and the like, but embodiments of the present disclosure are not limited thereto.

As some examples, the second optical portion OP2 may include an access body OP2_access for being connected with the whole portion WP.

Although FIG. 5b shows that the access body OP2_access has the shape of a bar and the number of access bodies OP2_access is two, this is merely for the convenience of description. That is, the shape and number of the access bodies OP2_access are not limited to those shown in FIG. 5b and can be freely modified.

In this case, the access body OP2_access may be connected with a connecting body WP_connect of the whole portion WP. In other words, the access body OP2_access of the second optical portion OP2 may be connected with the connecting body WP_connect of the whole portion WP, thereby forming a union. The specific method in which the access body OP2_access and the connecting body WP_connect are connected will be described later.

The whole portion WP performs refractive power correction or improvement, and the like, and is also connected to the second optical portion OP2 and serves to support and sustain the second optical portion OP2.

Although FIGS. 5a to 5e shows that the whole portion WP has a rectangular parallelepiped shape with rounded corners, embodiments of the present disclosure are not limited thereto, and as a matter of course, the shape of the whole portion WP may have a variety of shapes, such as a cube with rounded corners, a cylinder, or a cone.

The whole portion WP may include a basic lens WP_lens.

As some examples, the basic lens WP_lens may perform refractive power correction or improvement, and the like. In this case, the basic lens WP_lens may have various parameter values. The parameters of the basic lens WP_lens may include the type, focal length, refractive index, progression angle, diameter, thickness, and the like of the basic lens WP_lens, but embodiments of the present disclosure are not limited thereto. The types of the basic lens WP_lens may include a biconvex lens, a single-sided convex lens, a bending concave lens, and the like, but embodiments of the present disclosure are not limited thereto.

In this case, the whole portion WP may include a plurality of basic lenses WP_lens as well.

If described taking FIGS. 5d and 5e as an example, FIG. 5d shows a whole portion WP including one basic lens WP_lens, and FIG. 5e shows a whole portion WP including two basic lenses WP_lens. In other words, FIG. 5d shows that the whole portion WP includes the basic lens WP_lens on the top side of the whole portion WP, and FIG. 5e shows that the whole portion WP includes basic lenses WP_lens on each of the top and bottom sides of the whole portion WP. In this case, the parameter values of each of the plurality of basic lenses WP_lens included in the whole portion WP may be the same or different from each other.

The whole portion WP may include a connecting body WP_connect for being connected with the second optical portion OP2.

Although FIG. 5b shows that the connecting body WP_connect has a ¬ shape and the number of connecting bodies WP_connect is two, this is merely for the convenience of description. That is, the shape and number of the connecting bodies WP_connect are not limited to those shown in FIG. 5b and can be freely modified.

The whole portion WP may include a predetermined number of through-holes WP_hole. The through-holes WP_hole may perform the role of allowing surgical instruments for simple retinal surgery to be inserted. The through-holes WP_hole may be utilized as a space into which surgical instruments for simple retinal surgery can be inserted. Simple retinal surgery may include treatment of vitreous floaters, small-scale vitrectomy, treatment of minute retinal tears, and the like, but embodiments of the present disclosure are not limited thereto. In this case, the diameter of the through-holes WP_hole may be 2 mm or less, and the diameter of the surgical instruments to be inserted into the through-holes WP_hole may be 1 mm or less, but embodiments of the present disclosure are not limited thereto.

Although FIGS. 5a to 5c show that the through-holes WP_hole have an arc shape, and the number of through-holes WP_hole is two, this is merely for the convenience of description. That is, the shape and number of through-holes WP_hole are not limited to those shown in FIGS. 5b and 5c and can be freely modified.

Although not shown in FIGS. 5a to 5e, the functional intraocular lens IOL2 of the present disclosure may further include sealing buttons to be inserted into the through-holes WP_hole. The sealing buttons will be described in more detail later in FIG. 7.

Such a functional intraocular lens IOL2 has a novel effect of enabling additional mounting and replacement of the optical portion (second optical portion, OP2) after the whole portion WP including the basic lens WP_lens is inserted into the eye. That is, the functional intraocular lens IOL2 of the present disclosure can allow the second optical portion OP2 to be additionally mounted without extracting the intraocular lens itself after the whole portion WP including the basic lens WP_lens is inserted into the eye, and thus has the effect of enabling refractive power correction or the like to be performed with ease.

Moreover, the functional intraocular lens IOL2 additionally has the effect that the surgical procedure is simple and safer surgery is possible as there is no need to remove the previously inserted optical portion (first optical portion) when refractive power correction or the like is required, unlike the prefabricated intraocular lens IOL1 described above in FIGS. 2a to 4.

Meanwhile, the second optical portion OP2 and the whole portion WP may have shapes corresponding to each other so that they can be coupled to each other.

For example, the second optical portion OP2 may be formed with a curvature corresponding to the curvature of the basic lens WP_lens of the whole portion WP. In other words, the curvature of the second optical portion OP2 may be the same as or very similar to the curvature of the basic lens WP_lens of the whole portion WP.

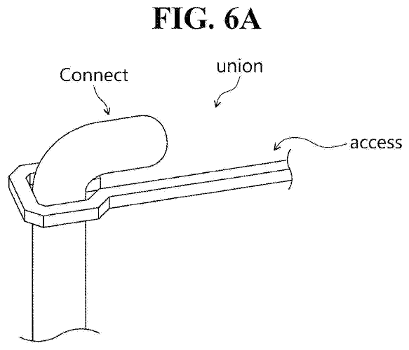

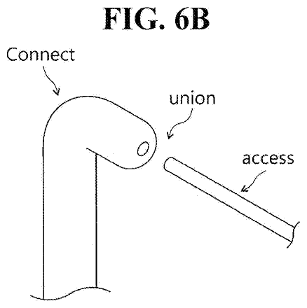

FIGS. 6a and 6b are diagrams for describing a connection method between an access body and a connecting body in accordance with some embodiments of the present disclosure.

Referring to FIGS. 6a and 6b, an access body access and a connecting body connect may be connected to each other and form a union.

The access body access shown in FIGS. 6a and 6b may refer to the access body OP1_access included in the prefabricated intraocular lens IOL1 shown in FIGS. 2a to 2d, the access body OPS_access included in the double-sided prefabricated intraocular lens IOL1_db shown in FIG. 4, the access body OP2_access included in the functional intraocular lens IOL2 shown in FIGS. 5a to 5e, and the like.

Similarly, the connecting body connect shown in FIGS. 6a and 6b may refer to the connecting body SP_connect included in the prefabricated intraocular lens IOL1 shown in FIGS. 2a to 2d, the connecting bodies SP_db_connect1 and SP_db_connect2 included in the double-sided prefabricated intraocular lens IOL1_db shown in FIG. 4, the connecting body WP_connect included in the functional intraocular lens IOL2 shown in FIGS. 5a to 5e, and the like.

FIG. 6a shows a connection method in the form in which the access body access is formed in the form of a gaff or hook and is wound around the connecting body connect, and FIG. 6b shows a connection method in the form in which the access body access is inserted and fixed inside the connecting body connect.

However, as a matter of course, the connection method between the access body access and the connecting body connect is not limited to that shown in FIGS. 6a and 6b.

FIG. 7 is a diagram for describing a sealing button and a through-hole in accordance with some embodiments of the present disclosure.

Referring to FIG. 7, a sealing button (hereinafter referred to as “SB”) may be connected to a through-hole hole. In other words, the sealing button SB may be inserted into the through-hole hole.

In this case, the through-hole hole may refer to the through-holes SP_hole included in the prefabricated intraocular lens IOL1 shown in FIGS. 2a to 2d, the through-holes SP_db_hole included in the double-sided prefabricated intraocular lens IOL1_db shown in FIG. 4, the through-holes WP_hole included in the functional intraocular lens IOL2 shown in FIGS. 5a to 5e, and the like.

As some examples, the sealing button SB may have a shape corresponding to the through-hole hole. For example, if the through-hole hole is in the shape of an arc, the sealing button SB may also be in the shape of a corresponding arc, but embodiments of the present disclosure are not limited thereto.

The present disclosure can prevent the vitreous body filling the retinal area behind the lens capsule from escaping through the through-hole hole via the sealing button SB inserted into the through-hole hole.

That is, the intraocular lenses IOL1, IOL1_db, and IOL2 of the present disclosure include through-holes hole to allow simple retinal surgery to be performed easily, but in this case, if the through-holes hole are left open, there may occur a problem that the vitreous body behind the lens capsule leaks to the outside through the through-holes hole.

Therefore, the intraocular lenses IOL1, IOL1_db, and IOL2 of the present disclosure have the effect of achieving both convenience in retinal surgery and prevention of vitreous body escape by opening the through-holes hole if retinal surgery is necessary and closing the through-holes hole off with the sealing button SB in other cases.

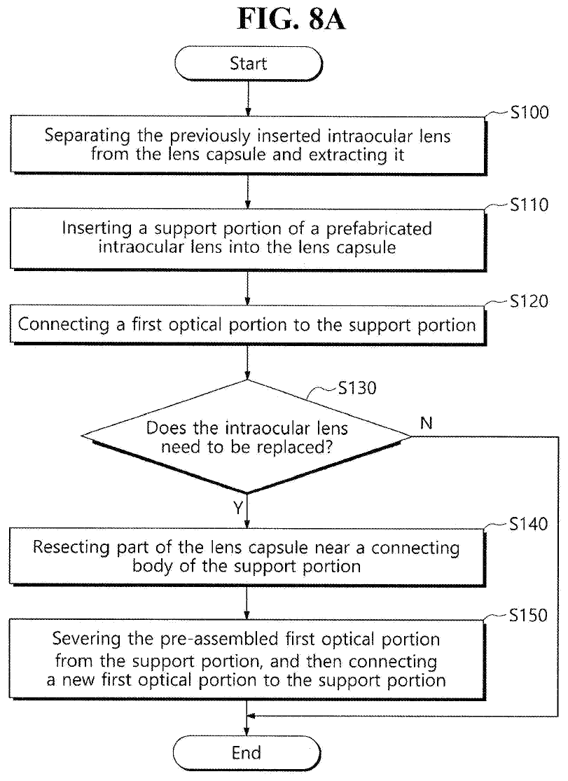

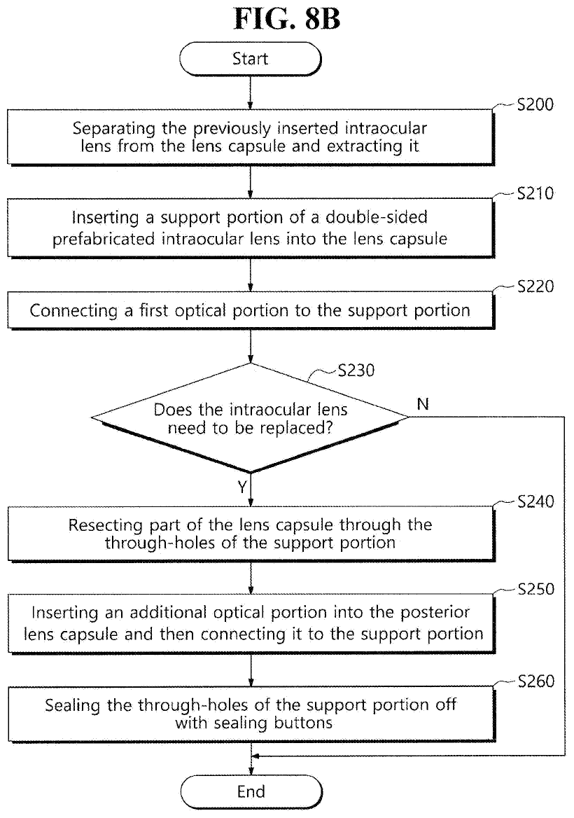

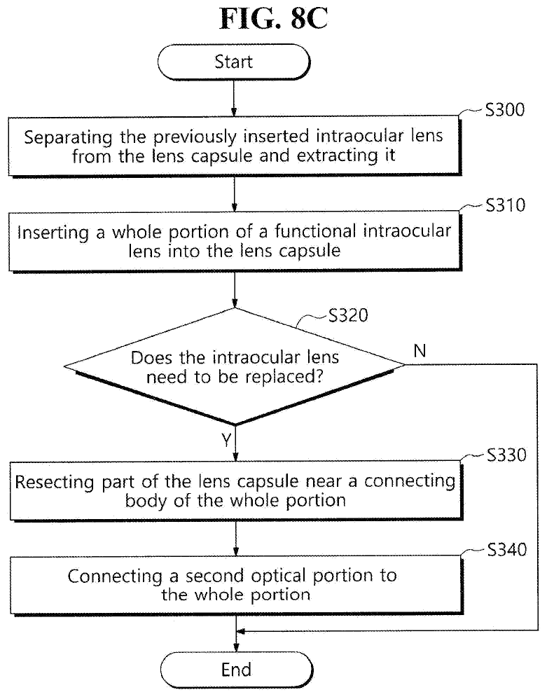

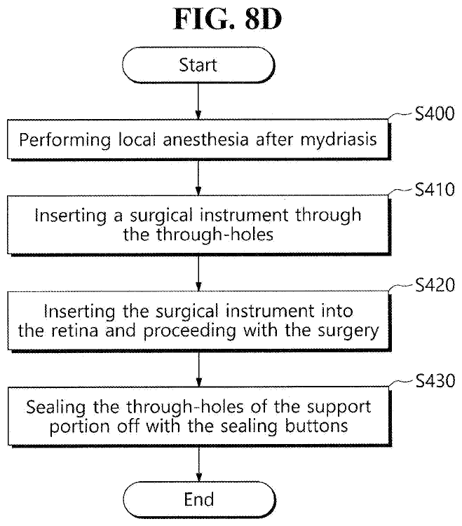

FIGS. 8a to 8c are flowcharts illustrating a method of inserting intraocular lenses into eyes in accordance with some embodiments of the present disclosure. The respective steps S100 to S340 of FIGS. 8a to 8c may be performed by humans and/or automated medical equipment, ophthalmic surgical equipment, surgical robots, or the like.

FIG. 8a is a flowchart showing a process of replacing the optical portion as necessary after inserting the prefabricated intraocular lens IOL1 shown in FIGS. 2a to 2d into the eye.

Referring to FIG. 8a, first, the previously inserted intraocular lens may be separated from the lens capsule and extracted (S100). At this time, the previously inserted intraocular lens may not be the intraocular lenses IOL1, IOL1_db, and IOL2 of the present disclosure but a generic intraocular lens (e.g., the conventional intraocular lens IOL of FIG. 1).

Next, the support portion SP of the prefabricated intraocular lens IOL1 may be inserted into the lens capsule (S110).

Next, the first optical portion OPT may be connected to the support portion SP (S120).

At this time, the access body OPT_access of the first optical portion OP1 and the connecting body SP_connect of the support portion SP may be connected to each other.

Next, it may be determined whether the prefabricated intraocular lens IOL1 needs to be replaced (S130). For example, the degree of error in the refractive power or the like of the prefabricated intraocular lens IOL1 may be determined.

As a result of the determination, if replacement is necessary, part of the lens capsule near the connecting body SP_connect of the support portion SP may be resected (S140).

Next, after severing the pre-assembled first optical portion OP1 from the support portion SP, a new first optical portion OP1 may be connected to the support portion (S150). At this time, the newly mounted first optical portion OP1 may be different from the existing first optical portion OP1 in parameter values. For example, the newly mounted first optical portion OP1 may be different from the existing first optical portion OP1 in at least one of the type, focal length, refractive index, progression angle, diameter, and thickness of the optical lens OP1_lens, but embodiments of the present disclosure are not limited thereto.

FIG. 8b is a flowchart showing a process of inserting the double-sided prefabricated intraocular lens IOL1_db shown in FIG. 4 into the eye and then connecting the additional optical portion as necessary.

Referring to FIG. 8b, first, the previously inserted intraocular lens may be separated from the lens capsule and extracted (S200). Step S200 may refer to substantially the same step as step S100 of FIG. 8a.

Next, the double-sided support portion SP_db of the double-sided prefabricated intraocular lens IOL1_db may be inserted into the lens capsule (S210).

Next, the first optical portion OP1 may be connected to the double-sided support portion SP_db (S220).

At this time, the first optical portion OP1 may be connected to the top side of the double-sided support portion SP_db. In this case, the access body OP1_access of the first optical portion OP1 and the connecting body SP_db_connect1 on the top side of the support portion SP may be connected to each other.

Next, it may be determined whether the double-sided prefabricated intraocular lens IOL1_db needs to be replaced (S230). For example, the degree of error in the refractive power or the like of the double-sided prefabricated intraocular lens IOL1_db may be determined.

As a result of the determination, if replacement is necessary, part of the lens capsule may be resected through the through-holes SP_db_hole of the double-sided support portion SP_db (S240).

Next, the additional optical portion OPS may be inserted into the posterior lens capsule and then be connected to the double-sided support portion SP_db (S250).

At this time, the access body OPS_access of the additional optical portion OPS and the connecting body SP_db_connect2 on the bottom side of the support portion SP may be connected to each other. In this case, the parameter values of the optical lens OPS_lens included in the additional optical portion OPS may be the same as or different from the parameter values of the optical lens OPT_lens of the first optical portion OPT.

Next, the through-holes SP_db_hole of the double-sided support portion SP_db may be sealed with the sealing buttons SB (S260).

As such, the present disclosure can prevent the vitreous body filling the retinal area behind the lens capsule from escaping through the through-holes SP_db_hole via the sealing buttons SB inserted into the through-holes SP_db_hole.

That is, the double-sided prefabricated intraocular lens IOL1_db of the present disclosure has the effect of achieving both convenience in mounting an additional optical portion or in retinal surgery and prevention of vitreous body escape by opening the through-holes SP_db_hole if the additional optical portion needs to be mounted or retinal surgery is required and closing the through-holes SP_db_hole off with the sealing buttons SB after the mounting of the additional optical portion or retinal surgery is completed.

FIG. 8c is a flowchart showing a process of inserting the functional intraocular lens IOL2 shown in FIGS. 5a to 5e into the eye and then connecting the second optical portion as necessary.

Referring to FIG. 8c, first, the previously inserted intraocular lens may be separated from the lens capsule and extracted (S300). Step S300 may refer to substantially the same step as step S100 of FIG. 8a and step S200 of FIG. 8b.

Next, the whole portion WP of the functional intraocular lens IOL2 may be inserted into the lens capsule (S310).

In this case, the whole portion WP may include a basic lens WP_lens, a connecting body WP_connect, through-holes WP_hole, etc.

Next, it may be determined whether the functional intraocular lens IOL2 needs to be replaced (S320). For example, the degree of error in the refractive power or the like of the functional intraocular lens IOL2 may be determined.

As a result of the determination, if replacement is necessary, part of the lens capsule near the connecting body WP_connect of the whole portion WP may be resected (S330).

Next, the second optical portion OP2 may be connected to the whole portion WP (S340).

At this time, the access body OP2_access of the second optical portion OP2 and the connecting body WP_connect of the whole portion WP may be connected to each other. In this case, the parameter values of the optical lens OP2_lens included in the second optical portion OP2 may be the same as or different from the parameter values of the basic lens WP_lens of the whole portion WP.

FIG. 8d is a flowchart illustrating a method of retinal surgery via through-holes and a method of sealing the through-holes in accordance with some embodiments of the present disclosure. The respective steps S400 to S430 of FIG. 8d may be performed by humans and/or automated medical equipment, ophthalmic surgical equipment, surgical robots, or the like.

Referring to FIG. 8d, first, local anesthesia may be administered after mydriasis (S400). In other words, local anesthesia may be administered after dilating the pupil of a subject.

Next, a surgical instrument may be inserted through the through-holes SP_hole, SP_db_hole, and WP_hole (S410). At this time, the diameter of the through-holes SP_hole, SP_db_hole, and WP_hole may be 2 mm or less, and the diameter of the surgical instrument may be 1 mm or less, but the embodiments of the present disclosure is not limited thereto.

Next, the surgical instrument may be inserted into the retina, thereby proceeding with the surgery (S420). The surgery to be performed at this time may include treatment of vitreous floaters, small-scale vitrectomy, treatment of minute retinal tears, and the like, but embodiments of the present disclosure are not limited thereto.

Next, the through-holes SP_hole, SP_db_hole, and WP_hole of the support portion may be sealed off with the sealing buttons SB (S430).

At this time, the sealing buttons SB may have a shape corresponding to the through-holes SP_hole, SP_db_hole, and WP_hole. For example, if the through-holes SP_hole, SP_db_hole, and WP_hole are in the shape of an arc, the sealing buttons SB may also be in the shape of a corresponding arc, but embodiments of the present disclosure are not limited thereto.

The present disclosure can prevent the vitreous body filling the retinal area behind the lens capsule from escaping through the through-holes SP_hole, SP_db_hole, and WP_hole via the sealing buttons SB inserted into the through-holes SP_hole, SP_db_hole, and WP_hole.

That is, the intraocular lenses IOL1, IOL1_db, and IOL2 of the present disclosure include through-holes SP_hole, SP_db_hole, and WP_hole to allow the simple retinal surgery described above to be performed easily, but in this case, if the through-holes SP_hole, SP_db_hole, and WP_hole are left open, there may occur a problem that the vitreous body behind the lens capsule leaks to the outside through the through-holes SP_hole, SP_db_hole, and WP_hole.

Therefore, the intraocular lenses IOL1, IOL1_db, and IOL2 of the present disclosure have the effect of achieving both convenience in retinal surgery and prevention of vitreous body escape by opening the through-holes SP_hole, SP_db_hole, and WP_hole if retinal surgery is necessary and closing the through-holes SP_hole, SP_db_hole, and WP_hole off with the sealing buttons SB in other cases.

The above description is merely an illustrative description of the technical idea of the present embodiments, and those of ordinary skill in the art to which the present embodiments pertain will be able to make various modifications and variations without departing from the essential characteristics of the embodiments. Therefore, the present embodiments are not intended to limit the technical idea of the present embodiments but to describe it, and the scope of the technical idea of the present embodiments is not limited by these embodiments.

The scope of protection of the present embodiments should be construed by the accompanying claims, and all technical ideas within the scope equivalent thereto should be construed as falling within the scope of the present embodiments.

What is claimed:

1. An intraocular lens comprising:
a first optical portion including an optical lens; and
a support portion configured to be coupled with the first optical portion,
wherein the first optical portion is formed to be detachable from the support portion in a state where the support portion is inserted into an eye of a user.

2. The intraocular lens of claim 1, wherein the first optical portion further comprises an access body,
the support portion comprises a connecting body,
the access body and the connecting body can be coupled with each other, and
the access body and the connecting body are the same in number, which is at least two or more.

3. The intraocular lens of claim 1, wherein the support portion and the first optical portion have shapes corresponding to each other so as to be coupled with each other.

4. The intraocular lens of claim 1, wherein the support portion comprises a predetermined number of through-holes.

5. The intraocular lens of claim 4, further comprising:
sealing buttons configured to be inserted into the through-holes formed in the support portion and perform a sealing function.

6. An intraocular lens comprising:
a first optical portion including an optical lens;
a support portion configured to be coupled with the first optical portion; and
an additional optical portion configured to be coupled to the support portion,
wherein the first optical portion and the additional optical portion are formed to be detachable from the support portion in a state where the support portion is inserted into an eye of a user, and
the first optical portion and the additional optical portion are coupled, respectively, to sides that are symmetrical to each other in the support portion.

7. An intraocular lens comprising:
a second optical portion including an optical lens; and
a whole portion configured to be coupled with the second optical portion and including a basic lens formed on a side in contact with the second optical portion,
wherein the second optical portion is formed to be detachable from the whole portion in a state where the whole portion is inserted into an eye of a user.

8. The intraocular lens of claim 7, wherein the second optical portion further comprises an access body,
the whole portion comprises a connecting body,
the access body and the connecting body can be coupled with each other, and
the access body and the connecting body are the same in number, which is at least two or more.

9. The intraocular lens of claim 7, wherein the whole portion comprises a predetermined number of through-holes.

10. The intraocular lens of claim 9, further comprising:
sealing buttons insertable into the through-holes formed in the whole portion.
